(19) United States
(12) Patent Application Publication
(10) Pub. No.: US 2022/0370763 A1
(43) Pub. Date: Nov. 24, 2022

(54) ANTEGRADE STEERING CATHETER AND METHOD FOR DELIVERING THERAPEUTIC DEVICES TO VAVLE SITES OF THE HEART

(71) Applicant: Synecor LLC, Durham, NC (US)

(72) Inventors: Richard S. Stack, Chapel Hill, NC (US); William L. Athas, Durham, NC (US); Kevin W. Johnson, Durham, NC (US)

(21) Appl. No.: 17/751,623

(22) Filed: May 23, 2022

Related U.S. Application Data
(60) Provisional application No. 63/191,903, filed on May 21, 2021.

(51) Int. Cl. A61M 25/01

(52) U.S. Cl. CPC A61M 25/0147; A61M 25/0136; A61M 25/0138; A61B 2017/00243

(57) ABSTRACT

A steering catheter includes an elongate shaft with an actively steerable distal part. An internal pull wire and an external pullwire extend through the shaft. The shaft has a first position in which the elongate shaft is generally straight and the external pullwire extends along the exterior of the distal portion of the shaft, a second position in which the shaft is curved and in which the external pullwire extends along the exterior of the distal portion of the shaft, and a third position in which the shaft is curved and in which the external pullwire extends laterally between laterally adjacent portions of the distal portion of the shaft, wherein the external pull wire locks the shaft in the curved position.

This application claims the benefit of U.S. Provisional Application No. 63/191,903, filed May 21, 2021, which is incorporated herein by reference.

Inventor: Richard S. Stack, William L. Athas, Kevin W. Johnson

### BACKGROUND

Transcatheter aortic valve replacement (TAVR) delivery systems are used to deliver replacement aortic valves to the aortic valve annulus using an intravascular approach. There are certain challenges associated with use of currently available TAVR delivery systems. In some patients, imprecise non-orthogonal placement of the TAVR device in the aortic valve annulus can cause paravalvular leak (PVL) and complete heart block (CHB). Impingement on the septum during valve expansion can create injury to the His bundle, resulting in the need for a permanent pacemaker. Precise positioning and orientation of the TAVR valve at the target site is highly desirable for avoiding such potential complications.

Commonly owned co-pending U.S. application Ser. No. 16/365,601 describes a transseptal delivery system for driving aortic valve therapeutic devices (AVTD's) such as TAVR delivery systems into place using a combination of pulling force, pushing force, steering force, and momentum. A related system that is used instead for transeptally driving transvascular mitral valve replacement (TMVR) valves or other mitral valve therapeutic devices into place is described in Applicant's co-pending application Ser. No. 16/396,677. Another co-pending U.S. application Ser. No. 16/578,373 describes a transseptal delivery system and method that may be used to deliver percutaneous ventricular assist devices, or other devices such as aortic valve therapeutic devices or mitral valve therapeutic devices to their target locations.

Commonly owned U.S. application Ser. No. 17/214,899 describes a method of using a system that is similar to that described in U.S. application Ser. No. 16/578,373 for delivering an aortic valve therapeutic device, such as a TAVR delivery system carrying a TAVR valve, to an aortic valve site using a modified approach to the aortic valve site. More particularly, the therapeutic device is introduced into the vasculature on the arterial side (e.g., via the right femoral artery “RFA”) vs the venous side as described in the prior co-pending applications. That system and method allow the TAVR delivery system to be precisely maneuvered coaxially into the center of the native or a prosthetic aortic valve, orthogonal to the aortic valve annulus and away from the subvalvular conduction system.

### BRIEF DESCRIPTION OF THE DRAWINGS

FIG. 1 is a perspective view of a steering catheter.

FIG. 1B is a side elevation view of the portion of the steering catheter encircled in FIG. 1A.

FIG. 1C is a perspective view of the distal tip section of FIG. 1B in the straight position.

FIG. 1D is a cross-section view of the steering catheter of FIG. 1A, taken along the plane designated 1D-1D in FIG. 1C.

FIG. 1E is similar to FIG. 1C, but further shows the protective covering on the external pullwire.

FIG. 1F is similar to FIG. 1E, but further shows the steering catheter in the curved position, but prior to deployment of the external pullwire.

FIG. 1G is similar to FIG. 1F, but further shows deployment of the external pullwire.

### DETAILED DESCRIPTION

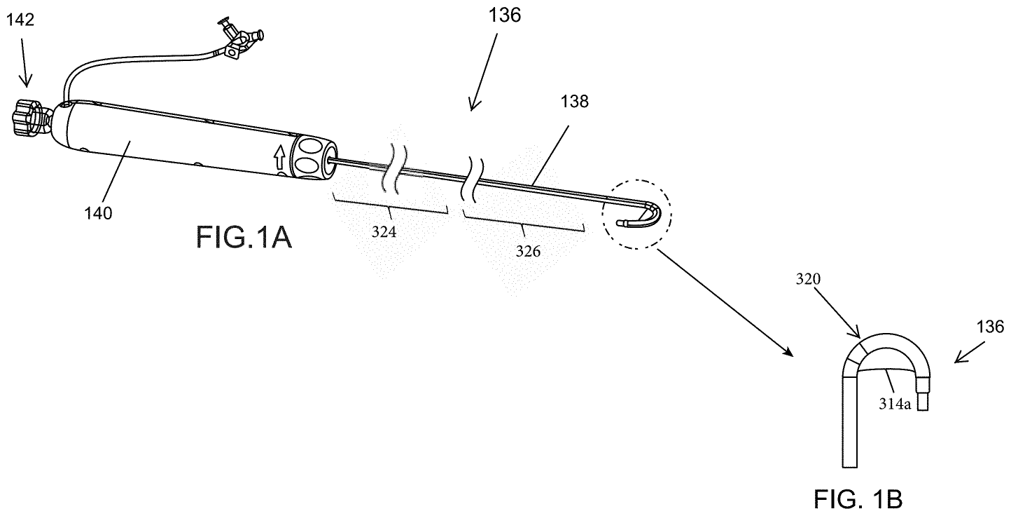

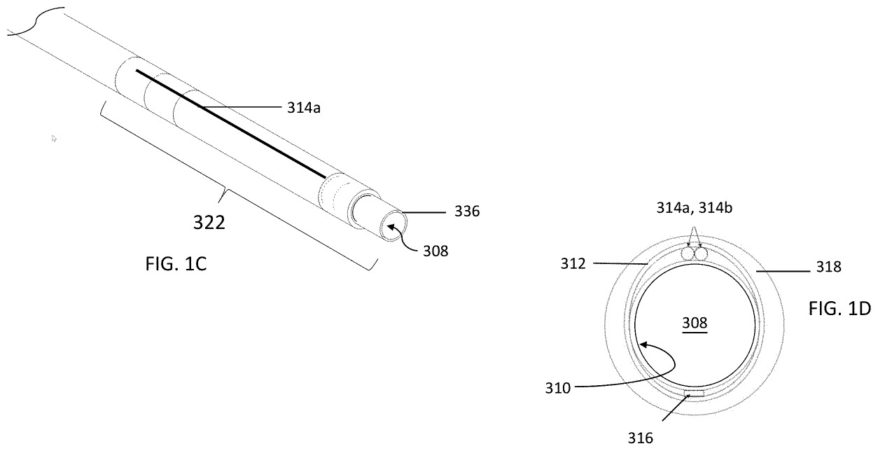

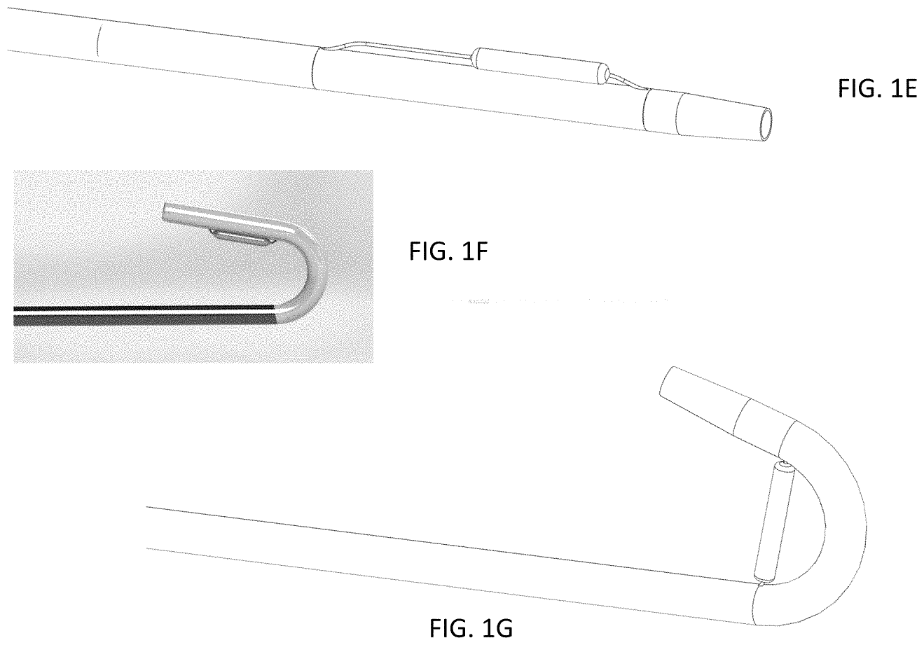

The presently disclosed steering catheter is designed to aid in the delivery of a TAVR device, other aortic valve therapeutic device or cardiac therapeutic devices to a location within the heart, such as at the aortic valve. A steering catheter has features similar to those of the “LVR” device described in U.S. application Ser. No. 16/365,601, U.S. application Ser. No. 16/578,373, and U.S. application Ser. No. 17/214,899, but it may differ in certain ways. In general, changes to its dimension and material properties, pullwire arrangement and handle may also be made without departing from the scope of the present invention. Additionally, the steering catheter differs from the LVR described in the referenced applications in ways that will be described with reference to FIGS. 1A-1E.

### Apparatus

Referring to FIGS. 1A-1D, steering catheter 136 includes an elongate catheter shaft 138 having a proximal handle 140 with a proximal access port 142 and a flush port. The shaft includes a lumen 308 accessible via the access port 142. This lumen extends to the distal tip of the shaft.

FIG. 1D is a cross-section view showing the shaft construction. As shown, a lubricious layer such as an extruded PTFE liner 310 lines the wall of the lumen 308, and a braid 312 covers the liner 310. Incorporated between the liner 310 and braid 312 are a pair of pullwires 314a, 314 and a return wire 316. The pull wires 314a, 314b are directly adjacent to one another. Their side-by-side positioning causes bending of the device along a bending plane P1 when tension on the wires is increased.

The return wire is positioned 180° from the pull wires as shown. It may have a rectangular diameter with the long edges oriented to cause the shaft to preferentially bend along bending plane P1. One of the pullwires 314a exits and then re-enters the shaft towards the shaft's distal end. This will be explained in the description of FIGS. 1B and 1C.

An outer jacket 318 of polymeric material (e.g., polyether block amide, “PEBA,” such as that sold under the brand name Pebax) 314 covers the braid 312. During manufacture of the shaft, the polymeric material is positioned over the braid and subjected to a reflow process to flow the polymeric material over the braid. The material properties of the polymeric material vary along the length of the shaft. This is discussed in detail in the prior applications referenced above.

The distal end of the shaft is moveable between the generally straight position shown in FIG. 1C, and an articulated position in which the distal end is formed into a curve, as shown in FIG. 1B. The parts of the shaft that are proximal to the curve 320 may be collectively referred to as the main body of the shaft. The prior applications include further details about the articulation angle, but the articulation angle of the catheter is preferably at least 90-100 degrees. The handle 140 (FIG. 1A) includes actuators to actuate the pull wires 314a, 314b to bend the shaft and to actuate the return wire 316 to return the distal end of the shaft to the generally straight configuration.

One of the pullwires 314a exits the sidewall of the shaft near the shaft's distal end, runs along the exterior of the shaft in a distal direction, and re-enters the shaft at the distal end of the shaft, while the other pull wire 314b does not exit the shaft at the distal end. The dual pull wire configuration advantageously allows articulation to the desired curvature and locking of the articulation in that curvature despite high loads experienced at the tip of the device during use.

The pull wire 314b that remains inside the shaft (“internal pull wire”) helps maintain the patency of the shaft's lumen during articulation, preventing the shaft from buckling or kinking despite the large degree of articulation as would likely happen if the construction used only the external pull wire.

The pullwire 314a that exits the shaft (the “external pull wire”) functions as a locking mechanism to lock the shaft in its articulated orientation, preventing the curve from opening when forces are exerted against the distal tip of the shaft. Another, related, feature is that when its tip is subjected to the forces described in the prior paragraph, the length of the pull wire 314b that is exposed outside the shaft 138 remains generally constant.

Note that the terms “pullwire” and “wire” are not intended to mean that the pullwires must be formed of wire, as these terms are used more broadly in this application to represent any sort of tendon, cable, or other elongate element the tension on which may be adjusted to change the shape of the catheter. Also, while the term “straight” is used to refer to the shape of the catheter shaft's distal portion in its non-articulated position, it should be pointed out that the catheter's inherent flexibility in the non-articulated position may cause it to bend under forces of gravity when held upright, or to curve when tracked over a curved cable or wire, or advanced into contact with another structure. The term “straight” thus should not be used to interpret this application or the corresponding claims as requiring that portion of the shaft to hold a straight shape.

Referring to FIGS. 1A-1D, one of the identified differences between the LVR described in the prior applications and the presently disclosed embodiments is that the handle 140, external pullwire 314a, and the internal pullwire 314b are configured so that the internal pullwire 314b may be actuated before the external pullwire 314a is actuated. Thus, for example, in use the pullwire 314b may be actuated to draw the distal part of the device 136 into a curved shape while the external part of the pullwire 314a remains in position extending along the exterior of the shaft 138 (rather than extending across the curve as shown in FIG. 1B). Then the pullwire 314a is actuated and subsequently moves to the position shown in FIG. 1B to increase the amount of curvature and/or to add force to retain the shaft 138 in the curved configuration. This two-phase actuation may be carried out by configuring the handle so that a user need only deploy a single actuation mechanism (e.g., rolling the knob on the handle), with the handle first increasing tension on the pullwire 314b, and then subsequently increasing the tension on the pullwire 314a. Alternatively, the handle may include two separate actuation mechanisms so that the user can manipulate one actuation mechanism to increase tension on the pullwire 314b and then manipulate another actuation mechanism to increase tension on the pullwire 314a.

Another distinction from the LVR described in the prior applications is a protective feature on the external pullwire. More specifically, the external pullwire 314a, which runs external to the shaft 138 in the distal part of the device 136 has a protective covering C, as shown in FIGS. 1E and 1F. This covering prevents contact between the external part of the pullwire 314a and surrounding tissue to avoid tissue trauma when the pullwire 314a is tensioned. The covering may be formed of any atraumatic material, such as a length of tubing formed of silicone or polymeric material.

### Methods of Use

Steering catheters of the type described herein may be used to deliver TAVR delivery systems or TMVR delivery systems to delivery sites within the heart. Once those delivery systems are delivered to the target sites using the steering catheter, the mechanisms of those delivery systems are activated in order to deliver the valve carried by the delivery system to the valve site.

In this section, a method of using a system of this type to deliver a TAVR delivery system carrying a TAVR device to the aortic valve site the heart will first be described. Afterwards, a method of using such the system to deliver a TMVR valve delivery system carrying a TMVR valve to its operative location within the heart will be described. It should be understood that these methods can be carried out using alternative steering catheters, and so it should be understood that this description is not limited to methods using the disclosed embodiments. Moreover, it should be understood that steps from the disclosed methods may be eliminated, and/or other steps may be added, without departing from the scope of the present invention. Because in these examples the steering catheter is used in an antegrade direction, the below examples refer to it as an “antegrade steering catheter” or “ASC.”

### First Example

This first example describes use of an antegrade steering catheter that has been placed into the left ventricle through the mitral valve as an accessory to guide the placement of transvascular aortic valve replacement (TAVR) valves and other aortic valve therapeutic devices

1. Place a 14 French access sheath in both the right femoral vein and the right femoral artery

2. Advance the ASC catheter and dilator through the venous sheath over a standard 0.035 steerable exchange guidewire (SEGW) to the inter-atrial septum.

3. Using intra-cardiac echo (ICE) for guidance, utilize a Brockenbrough style needle to enter the left atrium and then advance the SEGW into the left atrium followed by the ASC catheter.

4. Remove the dilator from the ASC catheter and replace it with a standard peripheral angioplasty balloon catheter and perform a standard atrial septostomy.

5. Advance the ASC catheter into the left atrium (LA) past the mitral valve orifice and retract both the balloon and SEGW into the ASC catheter.

6. Articulate the ASC catheter 90 degrees to the first position shown in FIG. 1F (where the external pullwire is not deployed to the position shown in FIG. 1G, but instead continues to follow the exterior surface of the shaft) and then withdraw it toward the mitral valve while applying counterclockwise torque until it enters the mitral valve annulus

7. Extend a small amount of the SEGW out of the end of the balloon and advance the tip of the balloon for 1.5 cm past the tip of the ASC catheter and inflate the balloon to between 1 and 2 atm to a diameter of 10-12 mm and advance the balloon and ASC catheter together to identify a clear pathway through the chordae tendineae and into the left ventricle in order to avoid chordal entrapment.

8. Articulate and torque the ASC toward the aortic valve (AV) and advance the SEGW retrograde through the aortic valve and around the aortic arch to the descending aorta.

9. Snare the SEGW from the arterial sheath and place the TAVR device over the SEGW, locking it onto the wire with the Tuohy Borst valve and advance it through the aorta to the level of the aortic valve as the guidewire is simultaneously retracted from the venous end of the ASC catheter.

10. Deploy the external pullwire to the position shown in FIG. 1G. Place forward pressure on the ASC catheter from the venous side to keep it anchored in the ventricular apex as the tip is articulated to guide the TAVR device into place through the aortic valve annulus in a central position, orthogonal to the annulus, by simultaneously pulling traction on the SEGW as the ASC is articulated in order to provide antegrade steering for precise placement of the TAVR device.

### Second Example

The second example describes placement of an antegrade steering catheter into the left ventricle through the aortic valve as an accessory to guide the placement of transvascular mitral valve replacement (TMVR) valves and other mitral valve therapeutic devices

1. Repeat steps 1-8 above to be able to advance the SEGW to the descending aorta without chordal entrapment, then snare the SEGW and withdraw it from the arterial sheath.

2. Remove the ASC catheter from the venous access sheath, leaving only the SEGW in the body as an “AV loop”, then reinstall the dilator and advance the ASC catheter retrograde across the aortic valve. Remove the dilator, and seat the articulated ASC catheter in the apex.

3. Place the TMVR device over the venous side of the SEGW and lock it onto the wire with the Tuohy Borst valve

4. Deploy the external pullwire to the position shown in FIG. 1G. Place forward pressure on the ASC catheter from the arterial side to keep it anchored in the ventricular apex as simultaneous traction is applied to the SEGW from the arterial side and the tip of the ASC catheter is articulated to guide the TMVR device across the interatrial septum into place through the mitral valve annulus in a central position, orthogonal to the annulus, in order to provide antegrade steering for precise placement of the TMVR device.

All prior patents and applications referenced herein are incorporated herein by reference.

We claim:

1. A steering catheter redirector comprising:
an elongate shaft having a tubular lumen, the shaft having a proximal portion and an actively steerable distal portion;
an internal pull wire and an external pull wire directly adjacent to the internal pull wire, wherein the external pull wire extends internally through the proximal portion of the shaft and longitudinally along the exterior of the distal portion of the shaft, wherein the shaft has a first position in which the elongate shaft is generally straight and the external pullwire extends along the exterior of the distal portion of the shaft, a second position in which the shaft is curved and in which the external pullwire extends along the exterior of the distal portion of the shaft, and a third position in which the shaft is curved and in which the external pullwire extends laterally between laterally adjacent portions of the distal portion of the shaft, wherein the external pull wire locks the shaft in the curved position;
a return wire extending longitudinally through the shaft, the return wire extending parallel to, and positioned 180 degrees from, the internal and external pull wires,
first and second actuators each moveable in a proximal direction and a distal direction, the first actuator operatively associated with the internal and external pull wires and the second actuator operatively associated with the return wire, the first and second actuators operatively coupled such that first and second actuators are moveable simultaneously in opposite ones of the proximal and distal directions, wherein the first actuator is moveable to a first actuator position to a second actuator position to move the steering catheter from the first position to the second position, and from the second actuator position to a third actuator position to move the steering catheter from the second position to the third position.
